(19) United States
(12) Patent Application Publication
(10) Pub. No.: US 2025/0268809 A1
(43) Pub. Date: Aug. 28, 2025

(54) BROAD-SPECTRUM PEPTOIDS FOR ORAL AND DENTAL HYGIENE, AND COMPOSITIONS AND METHODS OF USE THEREOF

(71) Applicant: Maxwell Biosciences, Inc., Austin, TX (US)

(72) Inventors: Beth Burnside, Santa Rosa Beach, FL (US); Sheetal Vali, Dallas, TX (US); Donald J. Treacy, Woodbine, MD (US); Edward M. Rudnic, Georgetown, TX (US)

(21) Appl. No.: 19/057,737

(22) Filed: Feb. 19, 2025

Related U.S. Application Data
(60) Provisional application No. 63/557,344, filed on Feb. 23, 2024.

(51) Int. Cl. A61K 8/64; A61K 9/00; A61K 9/06; A61K 9/08; A61K 9/20; A61K 38/08; A61P 31/04; A61P 31/10; A61P 31/22; A61Q 11/00; A61Q 17/00

(52) U.S. Cl. CPC A61K 8/64; A61K 9/0019; A61K 9/06; A61K 9/08; A61K 9/2072; A61K 38/08; A61P 31/04; A61P 31/10; A61P 31/22; A61Q 11/00; A61Q 17/005

(57) ABSTRACT

Broad spectrum peptoids for treatment or prevention of oral infections, and compositions and methods of use thereof, are described.

### RELATED APPLICATIONS

This application claims the benefit of U.S. Provisional Application No. 63/557,344 filed Feb. 23, 2024, the contents of which is incorporated by reference herein in its entirety.

### TECHNICAL FIELD

The present disclosure relates to methods for oral and dental hygiene using peptoid compounds.

### BACKGROUND

There is a need for improved treatment and prevention of infections in the oral cavity, the lips, and for other oral and dental infections. For example, there is a need for broad-spectrum oral treatments to address routine dental diseases and prevalent chronic diseases of the oral cavity, the lips, and for other oral and dental infections, particularly those that occur in absence of regular hygiene. Example environments where diagnostic testing of infections and/or regular hygiene may not be possible include, but are not limited to the battlefield, or in remote environments such as on submarines and other remote environments.

However, the development of new and improved anti-pathogenic compositions and methods of treatment and prevention of infections of the oral cavity, lips, and for other oral and dental infections remains challenging.

### SUMMARY

The present disclosure relates in several embodiments to peptoid compounds, compositions and method of use thereof for treating or preventing infections in the oral cavity, the lips, and for other oral and dental infections.

According to a first aspect, the present disclosure relates in several embodiments to compositions for use in preventing or treating an oral infection of a subject. The composition comprises one or more peptoid compounds in an amount of effective to prevent or treat the oral infection.

The compositions for use in preventing or treating an infection in the oral cavity, the lips, and for other oral and dental infections, may include the following details, which can be combined with one another in any combinations unless clearly mutually exclusive:
(i) The one or more peptoids may be selected from: a peptoid compound H-Ntridec-NLys-Nspe-Nspe-NLys-NH₂, a peptoid compound H-NLys-NSpe(p-Br)-NSpe(p-Br)-NLys-NSpe(p-Br)5-NSpe(p-Br)-NH₂, and a combination thereof, wherein each peptoid is in an amount of effective to prevent or treat the oral infection.
(ii) The oral infection may be an infection of the oral cavity, gums, lips, teeth, or any combination thereof.
(iii) The infection may an infection of a pathogen that has not been identified prior to the administration of the composition to the subject.
(iv) The infection may be a bacterial infection, a fungal infection, a viral infection, or any combinations thereof.
(v) The infection may be an infection of a pathogen selected from Herpes Simplex Virus-1 (HSV-1), Streptococcus mutans, Candida albicans, Porphyromonas gingivalis, and any combinations thereof.
(vi) The composition may be effective to prevent, decrease, or inhibit decrease a biofilm associated with the oral infection.
(vii) The composition may be formulated for topical administration, transdermal administration, transmucosal administration, subcutaneous administration, intramuscular administration, intravenous administration, or any combinations thereof, to the subject.
(viii) The composition may be formulated as a sublingual tablet, a gum, a lozenge, a thin film, a spray, a rinse, a toothpaste, an ointment, a creme, a balm, or any combinations thereof.
(ix) The effective amount may be from 1-1000 mg/day, 25-750 mg/day, 50-500 mg/day, or 100-400 mg/day.
(x) The composition may be formulated for administration one, two, three, or four times per day, once per week, once every two weeks, or once per month.

According to a second aspect, the present disclosure relates in several embodiments to a method of preventing or treating an oral infection in a subject. The method comprises administering a composition of the present disclosure, in an amount of effective to prevent or treat the oral infection.

### BRIEF DESCRIPTION OF THE DRAWINGS

The present disclosure may be further understood through reference to the attached figures in combination with the detailed description that follows.

FIG. 1 is the molecular structure of peptoid compound MXB-24,656.

FIG. 2 is the molecular structure of peptoid compound MXB-22,510.

FIG. 3 is the molecular structure of peptoid compound MXB-27,369.

FIG. 4 is the molecular structure of peptoid compound MXB-25,605.

FIG. 5 is the molecular structure of peptoid compound MXB-24,816.

FIG. 6 is the molecular structure of peptoid compound MXB-25,739.

FIG. 7 is a Table reporting example in vitro activity of peptoid compounds MXB-24,656 and MXB-22,510 against HSV-1, S. mutans, P. gingivalis, and C. albicans.

FIG. 8 is a graph reporting example time course and kill kinetics of peptoid compounds MXB-27,369, MXB-24,656 and MXB-22,510 against HSV-1.

FIG. 9 is a graph reporting example time course and kill kinetics of peptoid compounds MXB-27,369, MXB-24,656 and MXB-22,510 against Candida albicans.

FIG. 10 and FIG. 11 are graphs reporting example in vivo activity of peptoid compound MXB-22,510 against HSV-1. Mice were infected with HSV-1 in their lips on day 0. On day 2 post infection, lesions were treated with MXB-22,510 in PBS at indicated concentrations daily for 5 days. At the end of the experiment, lips and trigeminal nerves were excised, and HSV-1 DNA was quantified by QPCR relative to mouse beta-actin. Differences are significant, *, p<0.05; **p<0.005.

FIG. 12 is a Table summarizing example in vitro data of anti-bacterial properties of peptoids.

FIG. 13 is a Table summarizing example in vitro data of anti-fungal properties of peptoids.

FIG. 14 is a Table summarizing example in vitro data of activity against ESKAPE biofilms.

### DETAILED DESCRIPTION

The present disclosure relates to peptoid compounds for oral and dental hygiene. In some embodiments, the present disclosure relates to the use of peptoid compounds as broad-spectrum agents for treating or preventing infections in the oral cavity, the lips, and for other oral and dental infections. In some embodiments, the oral infection is an infection of the oral cavity, gums, lips, teeth, or any combinations thereof.

As described herein, in several embodiments, use of the peptoid compounds described herein for preventing or treating infections in the oral cavity, on the lips, and for other oral and dental infections offers advantages including but not limited to having antibacterial, antiviral and antifungal properties in one molecule, and the additional benefit of preventing and disrupting biofilms, both bacterial and fungal. The peptoid compounds described herein also offer other advantages, such as inducing neutrophils to express anti-inflammatory cytokines, promoting apoptosis of damaged cells and supporting healing. An additional advantage is that one can treat infections in the oral cavity, on the lips, and other oral and dental infections without the delay of diagnosing specific pathogens. Therefore, the pathogen and strain agnostic peptoid compounds described herein can act in both a preventative and treatment setting.

In some non-limiting examples, there is a need for improved treatment and prevention of infections in the oral cavity, on the lips, and for other oral and dental infections. In some embodiments, the present disclosure relates to the use of peptoids for treating dental caries, gingivitis, fungal infections and Herpes Simplex Virus. The peptoids described herein have activity as pathogen-agnostic Dental treatments to address routine dental diseases and prevalent chronic diseases of the oral cavity, the lips, and for other oral and dental infections, particularly those that occur in absence of regular hygiene. Example environments where regular hygiene may not be possible is in the battlefield, or in remote environments such as on submarines and other remote environments.

In some embodiments described herein, the peptoids compounds show good activity against pathogens that commonly infect sites including but not limited to, the oral cavity, lips, teeth and gums. Such pathogens include but are not limited to Herpes Simplex Virus-1 (HSV-1), Streptococcus mutans (Dental Caries), Candida albicans (Thrush) and Porphyromonas gingivalis, (major bacteria in chronic periodontitis). In addition, peptoids described herein demonstrate disruption of oral biofilms.

In some embodiments, the present disclosure relates to the use of peptoid compounds described herein as pathogen-agnostic prophylactics that prevent bacterial, fungal and viral wound infections, as well as the ability to disrupt and prevent biofilm formation, thus combating the threat of resistant and severe infections requiring extreme medical procedures. In some embodiments, the present disclosure relates to the use of broad-spectrum peptoids with the ability to prevent wound infections due to ESKAPE pathogens.

The terms “peptoid” or “peptoid compound” as used herein refers to a type of biomimetic molecule that is similar to peptides but differs in its structure. Peptoids are synthetic oligomers composed of N-substituted glycine units. Accordingly, peptoids are also known as poly-N-substituted glycine compounds. In contrast to peptides, which have a peptide bond between amino acids, peptoids have a N-substituted (or N-alkylated) amide bond. This structural difference gives peptoids unique properties compared to peptides. Peptoids can be designed and synthesized to mimic the functions of natural peptides but with enhanced stability and different chemical properties. Peptoid compounds may be cyclic or linear. Peptoids have been described, for example, in U.S. Pat. Nos. 8,445,632, 8,828,413, 9,315,548, 9,872,495, 9,938,321, and International Patent Application Publication No.'s WO2021046562, WO2020223581, WO2021127294, WO2023287570, WO2022120393, and WO2021231343, the disclosures of which are incorporated herein in their entireties.

For example, without limitation, in some embodiments a peptoid compound may have a formula:

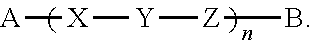

In such a compound, A can be selected from H and a terminal N-alkyl substituted glycine residue, where such an alkyl substituent can be selected from about C4 to about C20 linear, branched and cyclic alkyl moieties; n can be an integer selected from 1-3; B can be selected from NH2, and one and two N-substituted glycine residues, such N-substituents as can be independently selected from α-amino acid side chain moieties and structural/functional analogs thereof; and X, Y and Z can also be independently selected from N-substituted glycine residues, such N-substituents as can be independently selected from α-amino acid side chain moieties and structural/functional analogs thereof and proline residues. Such X-Y-Z periodicity can provide such a compound a certain amphipathicity. As would be understood by those skilled in the art, such structural and/or functional analogy can be considered in the context of any such α-amino acid side chain, N-substituent and/or a sequence of such N-substituted glycine residues, such structure and/or function including but not limited to charge, chirality, hydrophobicity, amphipathicity, helical structure and facial organization. Such analogs include, without limitation, carbon homologs of such side chain—such homologs as would be understood in the art, including but not limited to plus or minus 1 or 2 or more methylene and/or methyl groups.

A can be H, and B can be selected from one or two N-substituted glycine residues, such a selection as can reduce the hydrophobicity of such a compound, as compared to compounds of 3-fold periodicity. In certain such embodiments, X can be an NLys residue; n can be 2-3; and B can be two N-substituted glycine residues. Without limitation, such a compound can be of a formula:

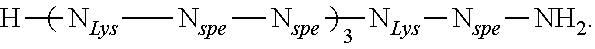

Regardless of identity of A, X and B, at least one of Y and Z can be a proline residue. X, Y and Z can be proline residues.

In certain other embodiments, A can be a terminal N-alkyl substituted glycine residue, with such an alkyl substituent as can be selected from about C6 to about C18 linear alkyl moieties. Regardless, B can be NH2, and n can be selected from 1 and 2. In certain such embodiments, A can be a terminal N-alkyl substituted glycine residue, with an alkyl substituent selected from about C6 to about C18 linear alkyl moieties. Regardless, B can be an NLys residue, and n can be 1.

In some embodiments, a peptoid compound may have a formula:

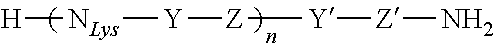

wherein n can be selected from 2 and 3; and Y, Z, Y′ and Z′ can be independently selected from N-substituted glycine residues, where such substituents can be independently selected from α-amino acid side chain moieties and carbon homologs thereof. Such Y′ and Z′ residues can be selected to provide such compound reduced hydrophobicity as compared to a compound of 3-fold periodicity. In certain such embodiments, at least one of X and Y can be a proline residue. Regardless, n can be selected from 2 and 3, and Y′ can be an N_{Lys} residue. In certain such embodiments, one or both X and Y can be proline residues. Without limitation, such a compound with reduced hydrophobicity can be of a formula:

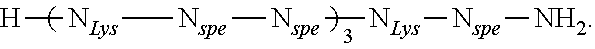

In some embodiments, a peptoid compound may have a formula:

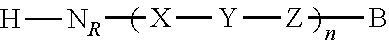

wherein B can be selected from NH₂ and X′; X, Y, Z and X′ can be independently selected from N-substituted glycine residues, where such substituents can be independently selected from α-amino acid side chain moieties and carbon homologs thereof; n can be an integer selected from 1 and 2; and R can be an N-alkyl substituent of such a glycine residue, as can be selected from about C₄ to about C₂₀ linear, branched and cyclic alkyl moieties. In some embodiments, n can be 2, and B can be NH₂. In some embodiments, n can be 1, and B can be X′. Accordingly, one or both of X and X′ can be NLys residues. Regardless, an alkyl substituent can be selected from about C₆ to about C₁₈ linear, branched and cyclic alkyl moieties, and X and X′ can be N_{Lys} residues. Without limitation, such a compound can be of a formula:

H-N_{tridec}-N_{Lys}-Nₛₚₑ-Nₛₚₑ-N_{Lys}-NH₂.

A peptoid may be a poly-N-substituted glycine compound comprising an N-terminus selected from H and an N-alkyl substituted glycine residue, where such an alkyl substituent can be selected from about C₄ to about C₂₀ linear, branched and cyclic alkyl moieties; a C-terminus selected from NH₂, one and two N-substituted glycine residues, such N-substituents as can be independently selected from a-amino acid side chain moieties and structural/functional analogs thereof; and 2 to about 15 monomeric residues between the N- and C-termini, each such residue as can be independently selected from proline residues and N-substituted glycine residues, said N-substituents independently selected from a-amino acid side chain moicties and structural/functional analogs thereof. Such monomers can be selected to provide such a compound a non-periodic sequence of monomers. As would be understood by those skilled in the art, such structural and/or functional analogy can be considered in the context of any such a-amino acid side chain, N-substituent and/or a sequence of such N-substituted glycine residues, such structure and/or function including but not limited to charge, chirality, hydrophobicity, amphipathicity, helical structure and facial organization. Such analogs include, without limitation, carbon homologs of such side chain—such homologs as would be understood by those skilled in the art, including but not limited to plus or minus 1 or 2 or more methylene and/or methyl groups.

The N-terminus of such a compound can be H; and the C-terminus can be selected from said one and two N-substituted glycine residues. A peptoid compound can comprise 2 to about 5 (X-Y-Z) non-periodic trimers. At least one of X, Y and Z in each of the trimers can be selected to interrupt 3-fold periodicity. Without limitation, at least one X in at least one said trimer can be an NLys residue. At least one of Y and Z in at least one such trimer can be a proline residue. The monomeric residues can comprise at least two non-consecutive of the same or repeat trimers, with at least one such residue therebetween to interrupt periodicity. At least one X in at least one such trimer can be an NLys residue, and at least one of Y and Z in at least one said trimer can be a proline residue.

The N-terminus of such a compound can be an N-alkyl substituted glycine residue, with an alkyl substituent selected from about C6 to about C18 linear alkyl moieties. A peptoid compound can comprise 2 to about 5 (X-Y-Z) non-periodic trimers. At least one of X, Y and Z in each of the trimers can be selected to interrupt 3-fold periodicity. The monomeric residues can comprise at least two non-consecutive of the same or repeat trimers, with at least one residue therebetween to interrupt periodicity. At least one X in at least one said trimer can be an NLys residue, and at least one of Y and Z in at least one said trimer can be a proline residue.

Various halogenated peptoids may be utilized in accordance with the teachings herein to make antiviral pharmaceutical compositions and treatments. These include, without limitation, various halogenated analogs of the foregoing peptoid compounds. These halogenated compositions may be halogenated in various ways. For example, these compounds may include any number of halogen substitutions with the same or different halogens. In particular, these compounds may include one or more fluoro-, chloro-, bromo- or iodo-substitutions, and may include substitution with two or more distinct halogens. In some embodiments, the use of one or two bromo-or chloro-substitutions may be used. The peptoids described herein may be halogenated at various locations, for example and without limitation para halogenation on the peptoids containing aryl rings, ortho-and meta-substitution, or perhalogentation.

The peptoids described herein may be alkylated, for example and without limitation terminal alkylation. For example and without limitation, the alkyl substituent may be selected from about C₆ to about C₁₈ linear alkyl moieties.

In some embodiments, a peptoid may have antibacterial activity, antifungal activity, antiviral activity, or any combination thereof.

Without intending to be bound by theory, the peptoid compounds described herein mimic the structures and functions of antimicrobial peptides, key constituents of the human immune system, to exert broad direct antibacterial, antiviral and antifungal activity and wound healing properties. Peptoids are structural variants of peptides, in which the side chain groups are appended to nitrogen (instead of carbon) to form an amphiphilic molecule with both hydrophobic and cationic features. This novel structure resists proteolysis to form a more stable compound in vivo with the same anti-pathogenic properties as natural peptides.

Without intending to be bound by theory, antiviral activity of a peptoid may be associated with its ability to pass through a viral membrane and to bind to viral DNA or RNA. Furthermore, also without intending to be bound by theory, the mechanism of action may also feature disruption of membranes of various pathogens, by preferentially interacting with the lipid phosphatidylserine, which is found on the outer leaflet of various pathogen membranes. Phosphatidylserine is not typically present on mammalian cell surfaces, allowing peptoid compounds to exhibit selectivity towards microbial cell types. The peptoid compounds described herein offer substantial pharmacological advantages over monoclonal antibodies and biological therapeutics: smaller size, low risk of off target effects, low manufacturing cost, anti-inflammatory properties, no cold chain requirement, high stability in vivo, and multiple mechanisms of action.

Various peptoid compounds may be utilized in accordance with the teachings herein to make pharmaceutical compositions and treatments, including without limitation the peptoid compounds described in the various patents and patent application publications described herein, which are incorporated herein in their entireties.

The peptoids described herein may be synthesized and provided by any suitable method known in the art, such as, for example and not by way of limitation, the method described in Example 1 of the present disclosure, or by methods described in the patents and patent application publications disclosed hercin.

Various counterions may be utilized in forming pharmaceutically acceptable salts of the peptoids disclosed herein. In some embodiments, pharmaceutically acceptable salts of the peptoids disclosed herein may include sodium or hydrochloride salts.

In some embodiments, the present disclosure extends to the preparation of prodrugs and derivatives of the peptoids of the invention. Prodrugs are derivatives which have cleavable groups and become by solvolysis or under physiological conditions the peptoid of the invention, which are pharmaceutically active. Such examples include, but are not limited to, choline ester derivatives and the like, N-alkylmorpholine esters and the like. In some embodiments, the peptoid compounds provided herein may be prepared e.g., in crystalline form and may be solvated or hydrated. Suitable solvates include pharmaceutically acceptable solvates, such as hydrates, and further include both stoichiometric solvates and non-stoichiometric solvates.

In some embodiments, the present disclosure relates to a composition for use in preventing or treating an infection in a wound in skin of a subject, the composition comprising one or more peptoids described herein. In some embodiments, the peptoid may be one or more of the peptoids described in Table 1 in the Examples of the present disclosure, including but not limited to peptoid compounds referred to herein as MXB-24,656, MXB-22,510, MXB-27,369, MXB-25,605, MXB-24,816, MXB-25,739.

It has been surprisingly found that not all peptoid compounds may be suitable for use as broad-spectrum, pathogen-agnostic, agents for use in treating or preventing infections in wounds.

In some embodiments, the peptoid compound for use as broad-spectrum, pathogen-agnostic, agent for use in treating or preventing infections in the oral cavity, the lips, and for other oral and dental infections is referred to herein as MXB-22,510, having a sequence H-Ntridec-NLys-Nspe-Nspe-NLys-NH₂, and having a molecular structure:

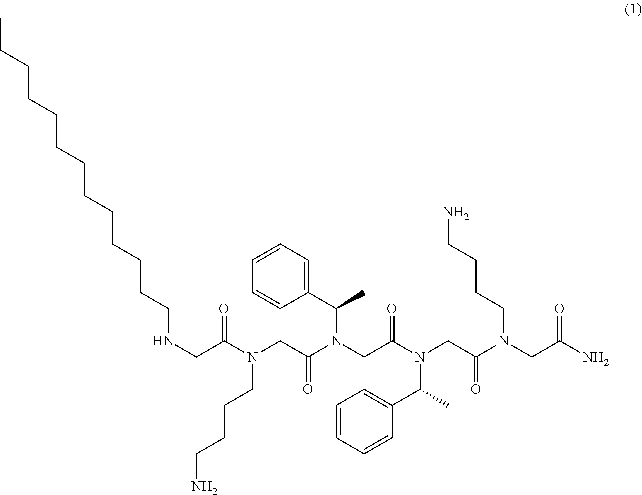 [SMILES: CCCCCCCCCCCCCNCC(=O)N(CCCCN)CC(=O)N(CC(=O)N(CC(=O)N(CCCCN)CC(N)=O)[C@H](C)c1ccccc1)[C@H](C)c1ccccc1]

In some embodiments, the peptoid compound for use as broad-spectrum, pathogen-agnostic, agent for use in treating or preventing infections in the oral cavity, the lips, and for other oral and dental infections is referred to herein as MXB-24,656 having a formula H-NLys-NSpe (p-Br)-NSpe(p-Br)-NLys-NSpe(p-Br)5-NSpe(p-Br)-NH2 and having a molecular structure:

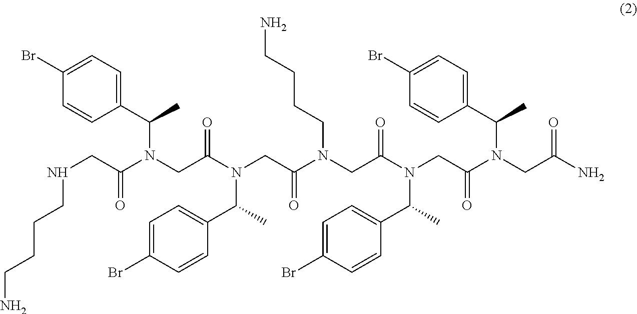 [SMILES: C[C@H](c1ccc(Br)cc1)N(CC(N)=O)C(=O)CN(C(=O)CN(CCCCN)C(=O)CN(C(=O)CN(C(=O)CNCCCCN)[C@H](C)c1ccc(Br)cc1)[C@H](C)c1ccc(Br)cc1)[C@H](C)c1ccc(Br)cc1]

As described in the Examples of the present disclosure, it has been unexpectedly found that MXB-22,510 and/or MXB-24,656 show surprisingly broad anti-pathogenic effects that are not observed in other peptoids tested. Accordingly, in several embodiments, the present disclosure relates to the use of the broad-spectrum anti-pathogenic peptoids MXB-22,510 and/or MXB-24,656 to treat or prevent a wide array of infections in the oral cavity, the lips, and for other oral and dental infections in a pathogen-agnostic manner. Compared with MXB-22,510 and/or MXB-24,656, other peptoids tested to date have shown significantly greater specificity of their anti-bacterial, anti-fungal, and/or anti-viral effects for treating pathogens commonly associated with infections in the oral cavity, the lips, and for other oral and dental infections.

In some embodiments, MXB-22,510 and/or MXB-24,656 have exceptionally broad activity and remarkable safety, when tested in various animal models and toxicology studies. Especially relevant is that MXB-22,510 and/or MXB-24,656 has shown activity against a broad range of bacteria, including gram positive and gram negative strains, as well as a broad range of fungi and even some viruses. MXB-22,510 and/or MXB-24,656 has also shown that it can prevent and disrupt biofilms, a major cause of concern for wound treatment. MXB-22,510 and/or MXB-24,656 have no significant activity against commensal microbiome bacteria, as demonstrated against human gut microbiome. The Examples of the present disclosure show example experimental data reporting the broad-spectrum activity of MXB-22,510 and/or MXB-24,656 against various pathogens.

Because of the broad-spectrum, pathogen-agnostic, activity of the peptoids, in some embodiments, the infection may be an infection of an unidentified pathogen at the time of administering the composition. In some embodiments, the composition of the present disclosure is adapted for administering to a subject for preventing or treating infections in the oral cavity, the lips, and for other oral and dental infections in the subject, prior to identifying, or confirming the identity of, the pathogen, or the presence of a pathogen, in the wound of the subject. In some embodiments, the infection may be an infection of one or more unidentified or unconfirmed bacteria, fungi, or viruses, or any combinations thereof.

In some embodiments, the infection may be a bacterial infection, a fungal infection, a viral infection, or any combinations thereof.

The one or more peptoids may be effective in simultaneously treating infections involving the one or more pathogens, including but not limited to one or more bacteria, fungi, viruses, or any combinations thereof.

In some embodiments, the one or more pathogens may show multi-drug resistant properties.

In some embodiments, the infection may be an infection of an oral pathogen selected from Herpes Simplex Virus 1, S. mutans, P. gingivalis, and C. albicans is and any combinations thereof.

The Examples of the present disclosure describe example data on activity of peptoids.

In some embodiments, the composition of the present disclosure may comprise an amount of the one or more peptoid compounds described herein effective to prevent, decrease, or inhibit decrease a biofilm associated with infections in the oral cavity, the lips, and for other oral and dental infections.

In some embodiments, the composition may be formulated for administration via one or more routes to the subject, in order to treat the infections in the oral cavity, the lips, and for other oral and dental infections. In some embodiments, the composition may be formulated for administration including but not limited to, topical administration, alone or in combination with systemic administration via intravenous, subcutaneous, or intramuscular injection to the subject.

In some embodiments, the present disclosure relates to a composition comprising one or more peptoid compounds described herein and one or more pharmaceutically acceptable excipients.

In some embodiments, the wound healing composition may be formulated for topical administration.

For example, and not by way of limitation, in some embodiments, a topical formulation of the present disclosure may be, or may comprise a sublingual tablet, a gum, a lozenge, a thin film, a spray, a rinse, a toothpaste, an ointment, a creme, a balm, or any combinations thereof. In some embodiments, the topical composition may be formulated for disposition into a bandage. In some embodiments, the topical formulation may be disposed in a bandage.

In some embodiments, the wound healing composition may be formulated for other or additional routes such as for administration to the subject's systemic circulation (c.g., the subject's bloodstream).

The compositions described herein may be administered to a subject via topical administration and/or one or more systemic routes, optionally in combination with topical application to the skin.

Accordingly, in some embodiments, the peptoid compounds of the present disclosure may be formulated in a composition suitable for administration to the subject via various routes to treat or prevent the infection. Such compositions can be prepared in a manner known in the pharmaceutical art. The peptoid compounds described herein can be formulated into pharmaceutically acceptable compositions and dosage forms for administration to a subject. In some embodiments, the present disclosure relates to a composition comprising an effective amount of a peptoid compound described herein for use in a method of treating a subject for an infection. In some embodiments, the present disclosure relates to the use of the peptoids described herein for the preparation of medicaments or as medicaments, that may be used for treating an infection.

The present disclosure provides pharmaceutical compositions comprising one or more peptoids and a pharmaceutically acceptable medium, such as an excipient, carrier, or the like. The peptoids described herein may be dissolved, suspended or disposed in various media. Such media may include, for example, various liquid, solid or multistate media such as, for example, emulsions, gels or creams. Such media may include liquid media, which may be hydrophobic or may comprise one or more triglycerides or oils. Such media may include, but is not limited to, vegetable oils, fish oils, animal fats, hydrogenated vegetable oils, partially hydrogenated vegetable oils, synthetic triglycerides, modified triglycerides, fractionated triglycerides, and mixtures thereof. Triglycerides used in these pharmaceutical compositions may include those selected from the group consisting of almond oil; babassu oil; borage oil; blackcurrant seed oil; black seed oil; canola oil; castor oil; coconut oil; corn oil; cottonseed oil; evening primrose oil; grapeseed oil; groundnut oil; mustard seed oil; olive oil; palm oil; palm kernel oil; peanut oil; rapeseed oil; safflower oil; sesame oil; shark liver oil; soybean oil; sunflower oil; hydrogenated castor oil; hydrogenated coconut oil; hydrogenated palm oil; hydrogenated soybean oil; hydrogenated vegetable oil; hydrogenated cottonseed and castor oil; partially hydrogenated soybean oil; soy oil; glyceryl tricaproate; glyceryl tricaprylate; glyceryl tricaprate; glyceryl triundecanoate; glyceryl trilaurate; glyceryl trioleate; glyceryl trilinoleate; glyceryl trilinolenate; glyceryl tricaprylate/caprate; glyceryl tricaprylate/caprate/laurate; glyceryl tricaprylate/caprate/linoleate;

glyceryl tricaprylate/caprate/stearate; saturated polyglycolized glycerides; linoleic glycerides; caprylic/capric glycerides; modified triglycerides; fractionated triglycerides; and mixtures thereof.

Various fatty acids may be utilized in the pharmaceutical compositions disclosed herein. These include, without limitation, both long and short chain fatty acids. Examples of such fatty acids include, but are not limited to, docosahexaenoic acid, caprylic acid, capric acid, lauric acid, butyric acid, and pharmaceutically acceptable salts thereof.

Generally, the peptoid compounds described herein are administered in a therapeutically effective amount. “Therapeutically effective amount” means the amount of a compound that, when administered to a subject for treating a disease, is sufficient to effect such treatment for the disease. The therapeutically effective amount of the peptoid compound may be determined by a physician, in the light of the relevant circumstances, including the condition to be treated, the chosen route of administration, the peptoid compound administered, the age, weight, and response of the individual subject, the severity of the subject's symptoms, and the like.

In some embodiments, the effective amount may be from 1-1000 mg/day, 25-750 mg/day, 50-500 mg/day, or 100-400 mg/day.

Moreover, these compositions may be administered in a single dose, multi-dose or controlled release fashion.

The term “administering”, “administered” and grammatical variants refers to the physical introduction of an agent to a subject, using any of the various methods and delivery systems known to those skilled in the art. Exemplary routes of administration include intravenous, intramuscular, subcutaneous, intraperitoneal, spinal or other parenteral routes of administration, for example by injection or infusion. The phrase “parenteral administration” means modes of administration other than enteral and topical administration, usually by injection, and includes, without limitation, intravenous, intramuscular, intraarterial, intrathecal, intralymphatic, intralesional, intracapsular, intraorbital, intraocular, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Non-parenteral routes include oral, topical, epidermal or mucosal route of administration, for example, intranasally, vaginally, rectally, sublingually or topically. Administering can also be performed, for example, once, a plurality of times, and/or over one or more extended periods.

In some embodiments, the administration may be one, two, three, or four times per day. In some embodiments, the administration may be once per week, once every two weeks, or once per month.

Formulation for topical administration may include, for example, dry powder formulation with a polymer to potentially extend residence time and drug release rate, spray on foam, topical gel, and an aqueous solution. These formulations can be dressed with a bandage or hemostatic gauze to maintain the formulation in place and to provide a protective barrier for healing. A powder formulation containing drug and polymer may be provided in a sachet or stick pack, where it could be administered directly or suspended in an aqueous solution for irrigation and administration. A simple powder formulation may be dissolved in an aqueous solution for irrigation and administration. A spray on foam formulation or a gel formulation may be administered via a small aerosol container. These product types provide for variable dosing and are suited for unpredictable conditions, i.e. windy, wet, varied temperatures, or may be used in a field hospital or tertiary care facility.

The pharmaceutical compositions disclosed herein may be manufactured as tablets, liquids, gels, foams, ointments or powders. In some embodiments, these compositions may be applied as microparticles or nanoparticles.

In some embodiments, intranasal compositions may comprise any pharmaceutically acceptable excipient, such as those approved in nasal spray formulations and listed in the Food and Drug Administration's Inactive Ingredient Database, or justifiable based on the Food and Drug Administration's Guidance for Industry: Nasal Spray and inhalation Solution, Suspension, and Spray Drug Products—Chemistry, manufacturing, and Controls Documentation. As would be understood by skilled persons, typically, the excipients used in intranasal formulations should be safe and compatible with nasal mucosa. Some common excipients used in intranasal products include buffers to maintain the pH of the formulation within an acceptable range, preservatives to prevent microbial contamination, surfactants to enhance drug absorption and distribution, stabilizers to maintain the stability of the formulation over time, solubilizers to improve the solubility of poorly soluble drugs, viscosity modifiers to control the viscosity of the formulation for better administration, and tonicity agents to adjust the osmolarity of the formulation to be close to that of nasal mucosa.

Compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for subjects, cach unit containing a predetermined quantity of active material calculated to produce the desired therapeutic effect, in association with a suitable pharmaceutical excipient. Typical unit dosage forms include prefilled, premeasured ampoules or syringes of the liquid compositions or pills, tablets, capsules or the like in the case of solid compositions. In such compositions, the peptoid compound is usually a minor component (c.g., from about 0.01% to about 50% by weight) with the remainder being various vehicles or carriers and processing aids helpful for forming the desired dosing form.

Liquid forms suitable for oral administration may include, without limitation, a suitable aqueous or nonaqueous vehicle with buffers, suspending and dispensing agents, colorants, flavors and the like.

Solid forms may include, without limitation any of the following ingredients, or compounds of a similar nature: a binder such as microcrystalline cellulose, gum tragacanth or gelatin; an excipient such as starch or lactose, a disintegrating agent such as alginic acid, Primogel, or cornstarch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. The peptoid compound in such compositions is typically a minor component, often being from about 0.05% to 10% by weight with the remainder being the injectable carrier and the like.

Transdermal compositions are typically formulated as a topical ointment or cream containing the peptoid compound, generally in an amount ranging from about 0.01 to about 20% by weight. When formulated as an ointment, the peptoid compound may be combined with either a paraffinic or a water-miscible ointment base. The peptoid compound may be formulated in a cream with, for example an oil-in-water cream base. Such transdermal formulations may include additional ingredients to enhance the dermal penetration of stability of the peptoid compounds or the formulation.

The peptoid compounds of the present disclosure can be administered by a transdermal device. Accordingly, transdermal administration can be accomplished using a patch either of the reservoir or porous membrane type, or of a solid matrix variety.

The peptoid compounds of the present disclosure can be administered subcutaneously, including, without limitation, the use of syringe and needle injection, autoinjectors, pen injectors, needle-free injectors, subcutaneous infusion, jet injectors, patch pumps, pump infusion sets, implantable devices, subcutaneous depots, subcutaneous sustained release formulations, or any combinations thereof. The most traditional and widely used method of subcutaneous administration involves using a syringe and needle. Autoinjectors are pre-filled devices that automatically inject a set dose of a pharmaceutical composition when pressed against the skin. Examples include, without limitation, EpiPen for epinephrine and various biologic medications. Similar to autoinjectors, pen injectors are pre-filled devices that allow patients to self-administer a specific dose of a pharmaceutical composition. They are user-friendly and may have features like dose adjustment. Needle-free injectors use high pressure to administer a pharmaceutical composition through the skin without using a needle. Subcutaneous infusion may involve using an infusion pump to deliver a continuous or intermittent flow of a pharmaceutical composition into the subcutaneous tissue. Jet injectors use a high-pressure stream of liquid to penetrate the skin and deliver a pharmaceutical composition into the subcutaneous tissue. Patch pumps may adhere to the skin and contain a reservoir of a pharmaceutical composition, and it is absorbed through the skin over a period of time. Pump infusion sets may include a cannula or needle that is placed under the skin for continuous pharmaceutical composition delivery. Implantable devices may be used to provide sustained release of a pharmaceutical composition subcutaneously. Implantable devices may be surgically implanted and can deliver a controlled dose of a pharmaceutical composition over an extended period.

Subcutaneous depot release refers to the administration of pharmaceutical compositions in a way that allows for sustained and controlled release of a pharmaceutical composition from a depot or reservoir located in the subcutaneous tissue. This method may be used to provide a prolonged therapeutic effect, reducing the frequency of dosing and improving patient compliance. In some embodiments, a subcutaneous depot release formulation may include, without limitation, a solution, a suspension, or biodegradable matrix, that is introduced (e.g., injected) into the subcutaneous tissue. The formulation then forms a depot, a localized reservoir of a pharmaceutical composition, beneath the skin. The subcutaneous depot formulation may release an active substance, e.g. a peptoid, gradually over an extended period.

A subcutaneous depot formulation may include, without limitation, biodegradable matrices, liposomal formulations, polymeric microspheres or nanoparticles, hydrogels, PLGA (poly(lactic-co-glycolic acid)) microparticles, implantable devices, or any combinations thereof. In biodegradable polymers or matrices, over time, the matrix breaks down, releasing a pharmaceutical composition in a controlled manner. Liposomes, which are lipid vesicles, can encapsulate a pharmaceutical composition and provide controlled release. Such liposomal formulations may be injected subcutaneously to create a depot of a pharmaceutical composition. Microspheres or nanoparticles made of biocompatible polymers can encapsulate a pharmaceutical composition and release it slowly over time. These particles can be suspended in a liquid formulation and injected into the subcutaneous tissue. Hydrogels are water-containing gels that can hold and release a pharmaceutical composition. Injectable hydrogels can form depots in the subcutaneous tissue. PLGA microparticles comprise PLGA, a biodegradable polymer commonly used to create microparticles for sustained drug release. PLGA microparticles can be injected subcutaneously to form a depot. Some subcutaneous depot release systems involve implantable devices, such as osmotic pumps or reservoirs. These devices are typically placed under the skin during a minor surgical procedure and provide controlled release of a pharmaceutical composition for an extended period.

Example formulations and methods of sustained release subcutaneous administration of the peptoids and pharmaceutical compositions thereof described herein include those described in the following references, the contents of all of which are incorporated herein in their entireties: Judy Senior, Michael L. Radomsky. (2000). Sustained-Release Injectable Products. Boca Raton: CRC Press; Thambi T, Li Y, Lee D S. Injectable hydrogels for sustained release of therapeutic agents. J Control Release. 2017 Dec. 10; 267:57-66. doi:10.1016/j.jconrel.2017.08.006. Epub 2017 Aug. 4. PMID: 28827094.; Chan Y P, Meyrueix R, Kravtzoff R, Nicolas F, Lundstrom K. Review on Medusa: a polymer-based sustained release technology for protein and peptide drugs. Expert Opin Drug Deliv. 2007 July; 4(4):441-51. doi:10.1517/17425247.4.4.441. PMID: 17683256.; Lou H, Feng M, Hageman M J. Advanced Formulations/Drug Delivery Systems for Subcutaneous Delivery of Protein-Based Biotherapeutics. J Pharm Sci. 2022 November; 111(11):2968-2982. doi:10.1016/j.xphs.2022.08.036. Epub 2022 Sep. 2. PMID: 36058255; Sequeira J A D, Santos A C, Serra J, Estevens C, Seiça R, Veiga F, Ribeiro A J. Subcutaneous delivery of biotherapeutics: challenges at the injection site. Expert Opin Drug Deliv. 2019 February; 16(2):143-151. doi:10.1080/17425247.2019.1568408. Epub 2019 Jan. 24. PMID: 30632401; Badkar A V, Gandhi R B, Davis S P, LaBarre M J. Subcutaneous Delivery of High-Dose/Volume Biologics: Current Status and Prospect for Future Advancements. Drug Des Devel Ther. 2021 Jan. 13; 15:159-170. doi:10.2147/DDDT.S287323. PMID: 33469268; PMCID: PMC7812053; Vaishya R, Khurana V, Patel S, Mitra A K. Long-term delivery of protein therapeutics. Expert Opin Drug Deliv. 2015 March; 12(3):415-40. doi:10.1517/17425247.2015.961420. Epub 2014 Sep. 24. PMID: 25251334; PMCID: PMC4605535; Remington's Pharmaceutical Sciences, 17th edition, 1985, Mack Publishing Company, Easton, Pa. The above-described components for intranasal, orally administrable, injectable subcutaneous, or topically administrable compositions are merely representative. Other materials as well as processing techniques and the like that are suitable for administering the peptoids and pharmaceutical compositions described herein are identifiable by skilled persons upon reading the present disclosure.

The peptoid compounds described herein can be administered in sustained release forms or from sustained release or controlled drug delivery systems, delivered via oral, intramuscular, subcutaneous, or transdermal route. A description of representative sustained release materials and description of delivery systems can be found in Remington's Pharmaceutical Sciences and Modern Pharmaceutics.

In some embodiments, the formulations described herein may include one or more chelation agents. In some embodiments, the chelation agent may be an efficacious anti-calculus agent including, but not limited to, one or more of zinc, hexametaphosphates, and diphosphonates. In some embodiments, the formulations described herein may include one or more chelation agents selected from aminopolycarboxylic acids, citric acid, edetate disodium anhydrous, edetate calcium disodium anhydrous citrate salts, sodium gluconate, transferrins, polymers, and any combinations thereof. In some embodiments, the aminopolycarboxylic acids may be selected from the group consisting of tetraxetan (DOTA), nitrilotriacetic acid (NTA), Ethylenediaminetetraacetic acid (EDTA or EDTA acid), ethylene glycol-bis(β-aminoethyl ether)-N,N,N′,N′-tetraacetic acid (EGTA or egtazic acid), 1,2-bis(o-aminophenoxy) ethane-N,N,N′,N′-tetraacetic acid (BAPTA), pentetic acid, diethylenetriaminepentaacetic acid (DTPA) nicotianamine, ethylenediamine-N,N′-bis(2 hydroxyphenylacetic acid) (EDDHA), Ethylenediamine-N,N′-disuccinic acid (EDDS), and any combinations thereof.

The following non-limiting formulation examples illustrate representative pharmaceutical compositions that may be prepared in accordance with the present disclosure.

Formulation 1—Tablets. A compound of the present disclosure may be admixed as a dry powder with a dry binder in an approximate 1:2 weight ratio. Additional diluent may be added as necessary, and a minor amount of magnesium stearate is added as a lubricant. The mixture is formed into 150-1500 mg tablets (50-500 mg of active compound per tablet) in a tablet press.

Formulation 2—Capsules. A peptoid compound described herein may be admixed as a dry powder with a starch diluent in an approximate 1:1 weight ratio. The mixture is filled into empty capsule shells (50-500 mg of peptoid compound per capsule).

Formulation 3—Liquid. A peptoid compound described herein (50-500 mg) may be admixed with sucrose (1.75 g) and xanthan gum (4 mg) and the resultant mixture may be blended, passed through a No. 10 mesh U.S. sieve, and then mixed in water. Sodium benzoate (10 mg), flavor, and color are diluted with water and added with stirring. Sufficient water may then be added to produce a total volume of 5 mL.

Formulation 4—Tablets. A peptoid compound described herein may be admixed as a dry powder with a dry gelatin binder in an approximate 1:2 weight ratio. A minor amount of magnesium stearate is added as a lubricant. The mixture is formed into 450-900 mg tablets (150-300 mg of active compound) in a tablet press.

Formulation 5—Injection. A peptoid compound described herein may be dissolved or suspended in a buffered sterile saline injectable aqueous medium to a concentration of approximately 0.1-5 mg/mL.

Formulation 6—Topical. Stearyl alcohol (250 g) and a white petrolatum (250 g) may be melted at about 75° C. and then a mixture of a peptoid compound described herein (1-100 g g) methylparaben (0.25 g), propylparaben (0.15 g), sodium lauryl sulfate (10 g), and propylene glycol (120 g) dissolved in water (about 370 g) may be added and the resulting mixture is stirred until it congeals.

Formulation 7—Intranasal. To prepare 1 L of a 25 mM phosphate buffer, dissolve 0.6 g of potassium phosphate dibasic and 2.93 g of potassium phosphate monobasic in 800 mL of deionized (DI) water. Second, slowly add 2 g of both glycerin and 2 g sorbitol while mixing with an overhead mixer. Stir until the solution is clear and free from undissolved particulates. Next, slowly add 50 g of hypromellose while stirring with an overhead mixer until dissolved for a 5% solution. Then, add 1 g of EDTA as a preservative for the multidose solution, and finally add 100 mg of the peptoid for a 0.01% (%w/v) solution. The solution is diluted to IL with DI water, the pH adjusted with a 1N NaOH or 1N HCl solution to a pH of 5.0-7.0 and stirred until a clear solution is obtained.

The peptoid may be included in the formulation over a range of 0.005%-5%. Alternate buffer agents include histidine buffer for pH control in the physiological range, and may be utilized over a molarity range of 10 mM-100 mM. Alternate viscosity increasing agents include, but are not limited to, carbomers, polyvinylpyrrolidone (PVP), hydroxyethylcellulose (HEC), and poloxamers, and may be present in a range of 2-10%. Osmolality increasing agents can also include, but are not limited to, sorbitol, sodium citrate, or dextrose, and may be included at 1-5%. Taste masking agents can include, but are not limited to, sucrose and/or other sugars and may be present at 1-5%. Preservatives may be included in the range of 0.05%-2%, and can also include, but are not limited to, benzalkonium chloride and sodium benzoate.

Formulation 8—Subcutaneous Injection. To prepare 1 L of a 25 mM phosphate buffer, dissolve 0.6 g of potassium phosphate dibasic and 2.93 g of potassium phosphate monobasic in 800 mL of deionized (DI) water. The solution is diluted to IL with DI water, the pH adjusted with a 1N NaOH or 1N HCl solution to a target pH of 6.5 (range 6.0-7.0) and stirred until a clear solution is obtained. Slowly add 1 g of peptoid and stir until completely dissolved for a target concentration of 1 mg/mL (0.1% w/v). This solution can be sterilized by using a 0.22 μm filter and stored in a sterile container with closure until use.

The peptoid may be included in the formulation over a range of 0.005%-5%. Alternate buffer agents include histidine buffer for pH control in the physiological range, and may be utilized over a molarity range of 10 mM-100 mM. Preservatives may be included in the range of 0.05%-2%, and can also include, but are not limited to, benzalkonium chloride and sodium benzoate.

Additional example peptoid formulations are described in Example 5.

In some embodiments, the compositions described herein may be formulated as mixtures of one or more peptoids. For example, these mixtures may comprise peptoids in various molar ratios, such as 0.01:0.99 to 0.99:0.01, or any ratio in between. In some embodiments, the effective amount may be from 1-1000 mg/day, with a preferred embodiment of 25-750 mg/day, or a more preferred embodiment of 50-500 mg/day, or an even more preferred embodiment of 100-400 mg/day.

In some embodiments, a composition may comprise a peptoid compound described herein in mixtures or combinations with other agents, such as known antibiotic, antifungal, or antiviral compounds. In some embodiments, the peptoid compounds of the present disclosure may act synergistically with the known antiviral compounds, so that the resulting composition demonstrates improved effectiveness.

In some embodiments, the present disclosure relates to a method of preventing or treating an infection in the oral cavity, the lips, and for other oral and dental infections. The method comprises administering a composition described herein, in an amount of effective to prevent or treat the infection.

In some embodiments, the subject may be a vertebrate animal. In some embodiments, the subject may be a mammal. In some embodiments, the subject may be a primate. In some embodiments, the subject may be a human. In some embodiments, the methods disclosed herein have veterinary applications and can be used to treat non-human animals, such as wild, domestic, or farm animals, including, but not limited to, cattle, sheep, goats, pigs, dogs, cats, and poultry. “Treating” or “treatment” of a infection refers, in some embodiments, to aiding in healing, ameliorating the infection (e.g., arresting or reducing worsening of the infection or at least one of the symptoms related to the infection). In some embodiments “treating” or “treatment” refers to ameliorating at least one physical parameter, which may not be discernible by the subject. In some embodiments, “treating” or “treatment” refers to modulating the infection, either physically, (c.g., stabilization of a discernible symptom), physiologically, (c.g., stabilization of a physical parameter), or both.

“Preventing” or “prevention” as used herein refers to a reduction in risk of acquiring a infection (e.g., causing at least one of the clinical symptoms of the infection not to develop in a subject not yet exposed to or predisposed or susceptible to the infection, and not yet experiencing or displaying symptoms of the disease).

In some embodiments, an infection may be a viral infection, a bacterial infection, a fungal infection, or any combination thereof.

In some embodiments, a compositions and methods described herein may be combined with other compositions and methods, including known oral hygiene or dental hygiene care active ingredients, compositions and methods, either in the same composition, or administered separately. In some embodiments, the compositions and methods of the present disclosure may act synergistically with the other active ingredients, compositions and methods, so that the resulting compositions and/or methods demonstrate improved effectiveness.

In some embodiments, the compositions and methods described herein may be useful for applications as oral or dental hygiene care products for example as consumer oral or dental hygiene care products, such as over-the counter products, or products prescribed by a healthcare professional. In some embodiments, the compositions and methods described herein may be useful for applications in extreme environments such as in the battlefield, for military use, or in emergency scenarios, such as in first aid kits, care for home use, for clinical use, for first responder uses, and the like.

In some embodiments, the compositions described herein are non-toxic to human cells, show improved tolerability, improved efficacy, or any combinations thereof, compared to previously existing compositions and products for treating or preventing infections.

### EXAMPLES

The present examples are provided for illustrative purposes only. They are not intended to and should not be interpreted to encompass the full breadth of the invention.

### Example 1. Preparation of Peptoid Compounds

The peptoid compounds listed in Table 1 were prepared using a sub-monomer protocol, on Rink Amide MBHA Resin. Example sub-monomer protocols are described in Zuckermann, R. N., Kerr, J. M., Kent, S. B. H., & Moos, W. H. (1992) J. Am. Chem. Soc., 114, 10646-10647 and in U.S. Pat. Nos. 8,445,632 and 6,887,845, the entireties of which are incorporated herein by reference. The starting reagents are bromoacetic acid and a small set of primary amines that are readily available commercially. The crude peptoid products were then cleaved from the resin and sidechain protective groups were removed in one step by acidolysis. The resulting residue was then resolubilized and lyophilized twice to produce peptoids as a dry powder. The peptoid products were then purified by HPLC to produce peptoids in powder form, with hydrochloride as the counter ion. Peptoid compounds were stored as dry powder at −20° C. and protected from light prior to preparation of stock solutions.

TABLE 1
Peptoid compounds tested against various pathogens

| Peptoid | | Molecular | | |
|---|---|---|---|---|
| compound | | weight without | Molecular | |
| name | Peptoid Sequence | HCl salt (g/mol) | Formula | Counter Ion |
| MXB-24,656 | H-NLys-NSpe(p-Br)- | 1233.78 | C₅₂H₆₇Br₄N₉O₆ | Hydrochloride |
| | NSpe(p-Br)-NLys-NSpe(p- | (1343.15 with | | |
| | Br)₅-NSpe(p-Br)-NH₂ | HCl salt) | | |
| MXB-22,510 | H-Ntridec-NLys-Nspe-Nspe- | 835.19 | C₄₇H₇₈N₈O₅ | Hydrochloride |
| | NLys- NH₂ | (944.56 with | | |
| | | HCl salt) | | |
| MXB-27,369 | H-Ndec-NLys-Nspe-Nspe(p- | 1273.31 | C₆₄H₉₂Br₂N₁₀O₇ | Hydrochloride |
| | Br)-NLys-Npse- Nspe(p-Br)- | (1382.68 with | | |
| | NH₂ | HCl salt) | | |
| MXB-25,605 | H-Ndodec-NLys-Nspe-Nspe- | 821.17 | C₄₆H₇₆N₈O₅ | Hydrochloride |
| | NLys-NH₂ | (930.53 with | | |
| | | HCl salt) | | |
| MXB-24,816 | H-Ntetradec-NLys-Nspe- | 849.22 | C₄₈H₈₀N₈O₅ | Hydrochloride |
| | Nspe-NLys-NH₂ | (958.58 with | | |
| | | HCl salt) | | |
| MXB-25,739 | H-Ndec-NLys-Nspe-Nspe(p- | 1401.49 | C₇₀H₁₀₄Br₂N₁₂O₈ | Hydrochloride |
| | Br)-NLys-Nspe-Nspe(p-Br)- | (1547.32 with | | |
| | NLys-NH₂ | HCl salt) | | |

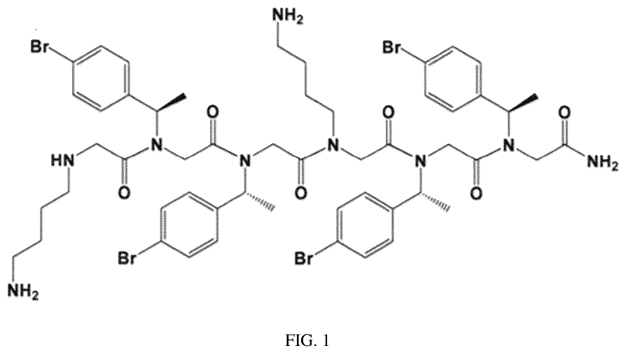

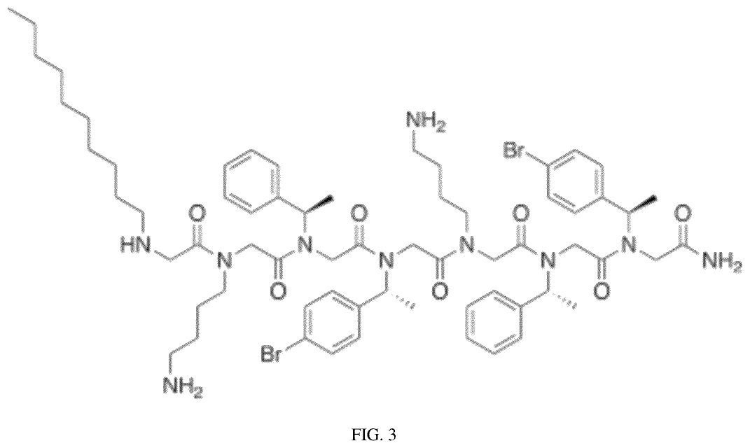

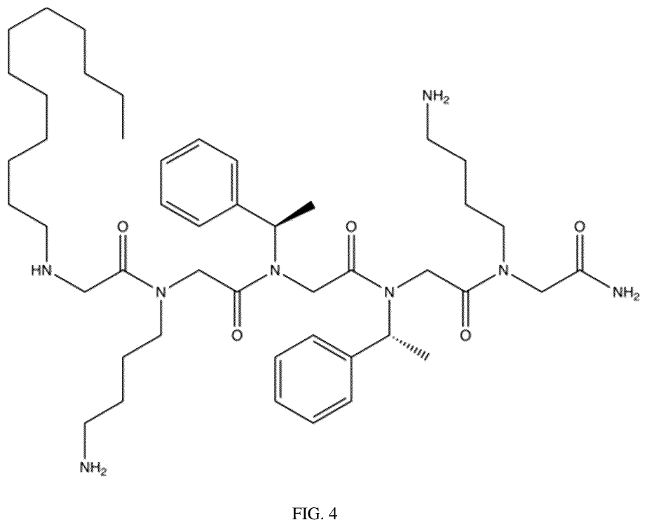

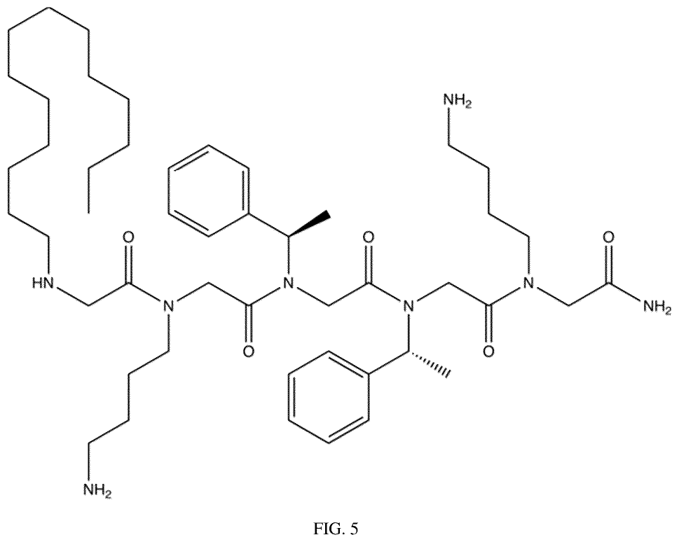

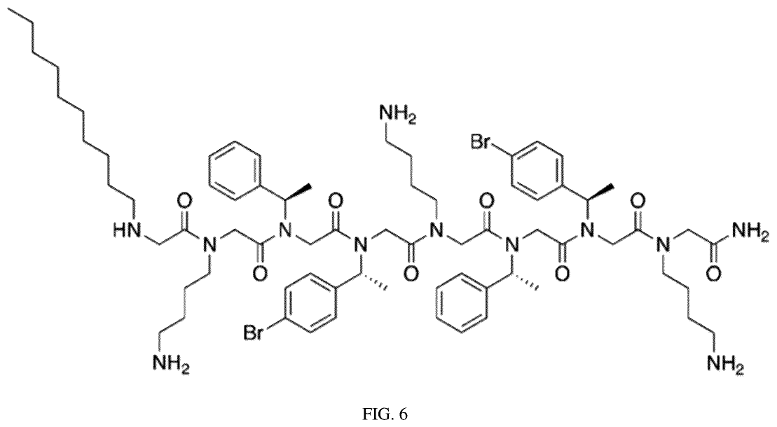

Chemical structures of the peptoid compounds listed in Table 1 are shown in FIG. 1-FIG. 6.

An initial stock concentration of each peptoid compound was prepared in tubes at 2 mg/ml in phosphate-buffered saline (PBS) pH 7.4 (Gibco; cat no. 10010023). Initial dissolution of lyophilized peptoid compound powders to create a stock solution was performed by gentle mixing by inverting the stock solution tube several times), followed by checking for turbidity, precipitation, or aggregate before proceeding to the next steps. If gentle inversion was insufficient to achieve a solution, the stock solution tube was briefly vortexed. The stock solution was then checked for any undissolved particulate, aggregates, or precipitation before proceeding to the next step. If gentle inversion and vortexing was insufficient to achieve a solution, the stock solution tube was briefly sonicated for 15-60 seconds. The stock solution was then checked again for any undissolved particulate, aggregates, or precipitation before proceeding to the next step. If turbidity, precipitation, or aggregate was observed at the initial stock concentration, the initial stock concentration was solubilized by diluting further in PBS to 1 mg/ml. Aliquots of the stock solutions were dispensed in polypropylene vials, protected from light, and stored at −20° C. or −80° C. prior to use.

Before testing, the aliquots were carefully observed for any signs of turbidity, precipitation or aggregate during sample preparation and were mixed, vortexed, or sonicated as needed.

### Example 2. In Vitro Activity of Peptoid Compounds Against Oral Pathogens

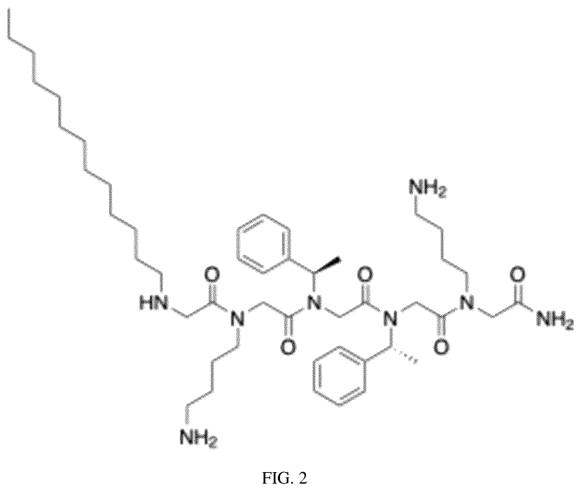

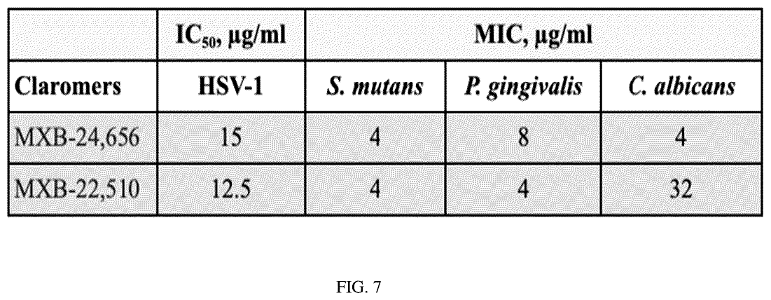

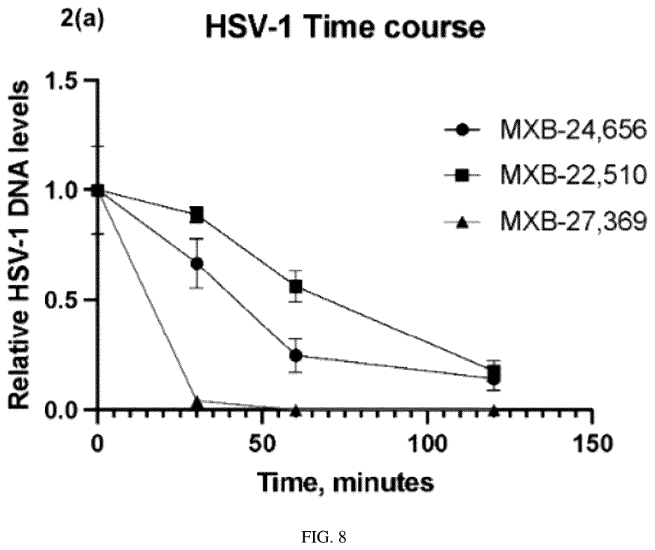

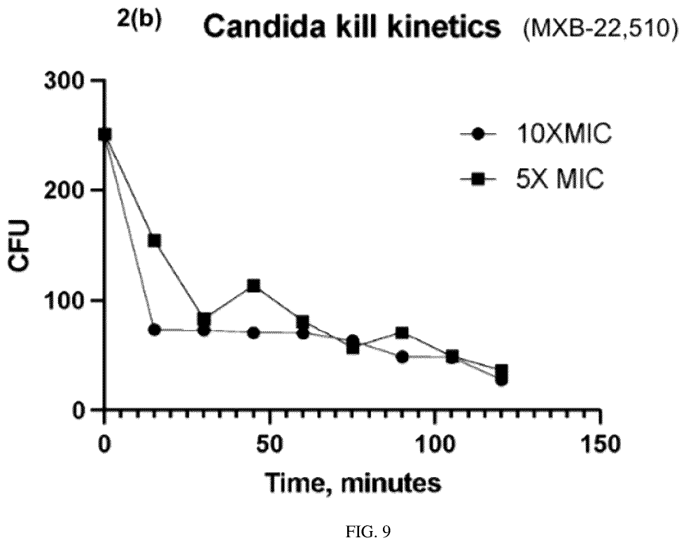

The activity of peptoid compounds MXB-24,656 and MXB-22,510 against four types of oral pathogens: Herpes Simplex Virus 1, S. mutans, P. gingivalis, and C. albicans is shown in FIG. 7. MXB-24,656 and MXB-22,510 demonstrate the most potent activity against all four pathogens to date. Time course analysis shows inactivation of HSV-1 as short as 30 minutes at 20 μg/ml (Diamond, Gill; Molchanova, Natalia; Herlan, Claudine; Fortkort, John A.; Lin, Jennifer S.; Figgins, Erika; Bopp, Nathen; Ryan, Lisa K.; Chung, Donghoon; Adcock, Robert Scott; Sherman, Michael; Barron, Annelise E. Pharmaceuticals (2021), 14(4), 304CODEN: PHARH2; ISSN: 1424-8247. (MDPI AG) and against C. albicans within 15 minutes. (FIGS. 2a and 2b of Diamond et al., Pharmaceuticals, 2022, are reproduced herein as FIG. 8 and FIG. 9, respectively).

### Example 3. In Vivo Efficacy of MXB-22.510 Against Herpes Simplex Virus-1 (HSV-1) in the Herpes Labialis Lip Scarification Mouse Model

In Vivo Efficacy of MXB-22,510 against Herpes Simplex Virus-1 (HSV-1) was studied in the herpes labialis lip scarification mouse model.

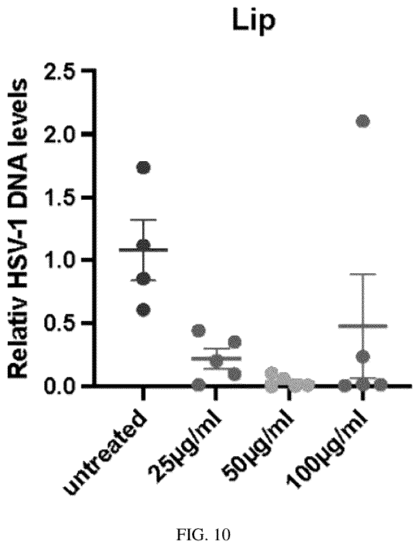

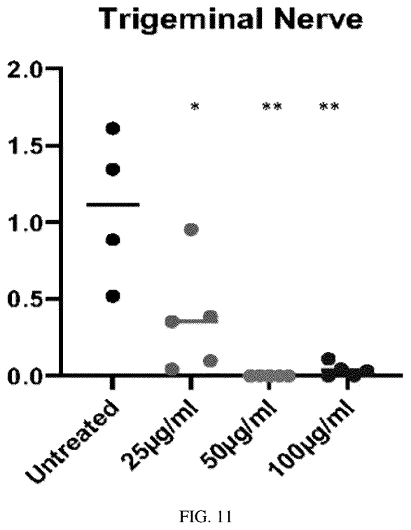

BALB/c mice were infected with HSV-1 strain 294.1. On day 2 post-infection, lips were treated with 0, 20, 50 and 100 μg/ml MXB-22,510 daily until day 6. At 50 μg/ml there were no lesions compared with control (0) and 20 μg/ml. DNA analysis of the excised lips and trigeminal nerve confirmed the dose-dependent activity of the peptoid in vivo in treating these herpes lesions. See FIGS. 10 and 11.

A decrease in lesion severity at the highest concentration, with almost no lesions visible by day 5 compared to buffer alone. In addition, prevention of HSV-1 from translocating to the trigeminal nerve, thus preventing latent viral infection.

### Example 4. In Vitro Activity of Peptoid Compounds Against Bacterial and Fungal Pathogens

Peptoid compounds have also demonstrated disruption of mixed species biofilms in a fluorescence microscopy assay with interkingdom biofilm of C. albicans and S. mutans grown on saliva-coated plastic. Biofilms were treated for 48 hours with 20 μg/ml MXB-22,510, followed by imaging with live-dead stains.

Peptoids in test groups A-F in Table 1 of Example 1 were tested in vitro for activity against several bacterial and fungal pathogens.

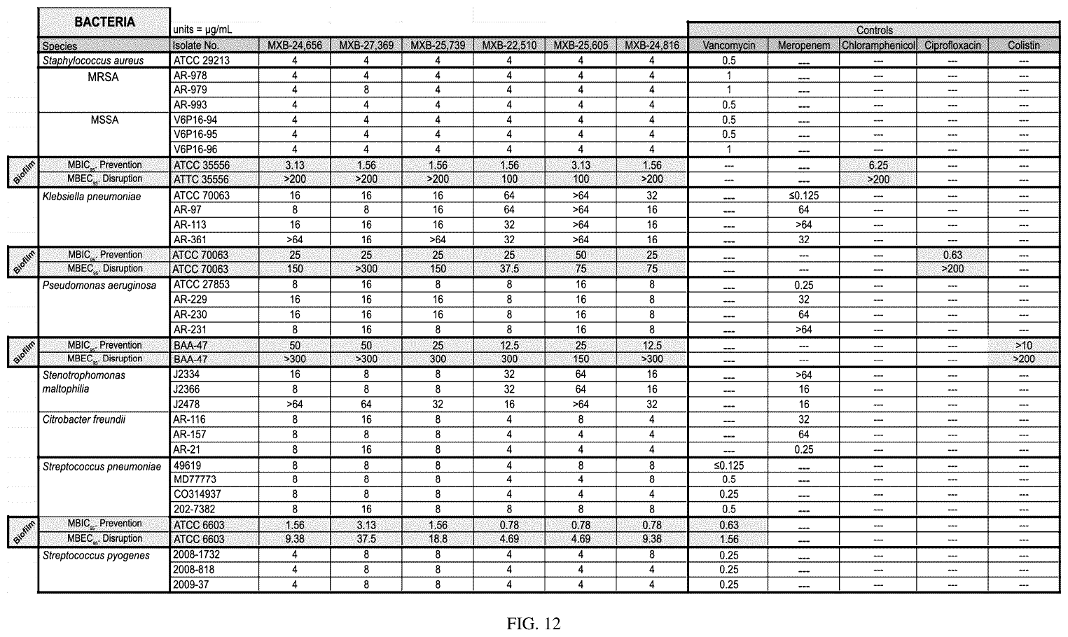

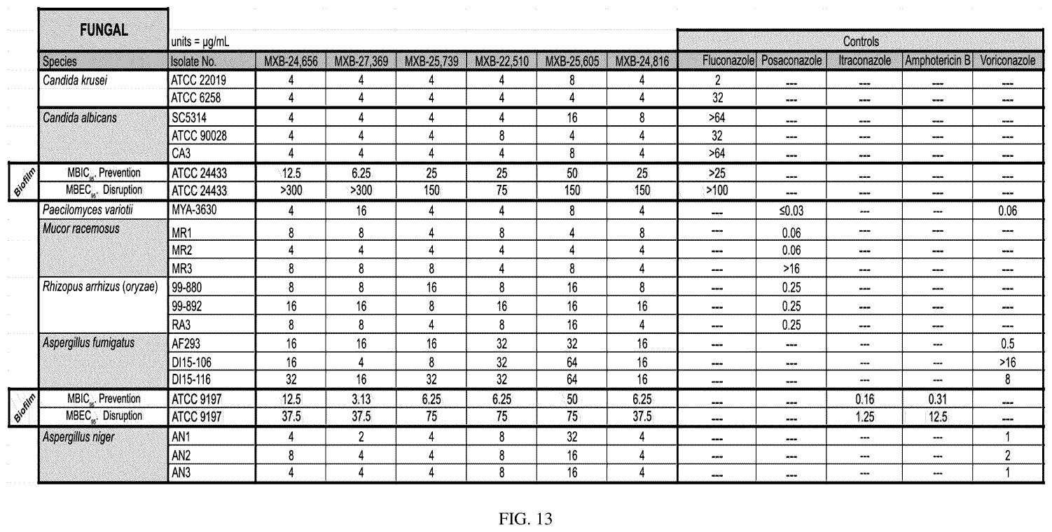

The results of anti-bacterial and anti-fungal activity for the tested peptoids is shown in FIG. 12 and FIG. 13, respectively.

In the Tables shown in FIG. 12 and FIG. 13, “MIC” refers to minimum inhibitory concentration, “MBIC” refers to minimum biofilm inhibitory concentration, and “MBEC” refers to minimum biofilm eradication concentration.

The peptoid compounds show in vitro activity against multiple bacterial and fungal pathogens for example but not limited to Staphylococcus aureus including both MRSA: methicillin-resistant Staphylococcus aureus, and MSSA: methicillin-susceptible Staphylococcus aureus and Aspergillus. As seen in FIG. 12 and FIG. 13, the peptoids tested achieved an MIC range of 4 μg/mL to 32 μg/mL depending on the species and strain tested. In addition, peptoids exert activity against biofilms, shown in FIG. 12 and FIG. 13.

Peptoids show the inhibition of biofilm formation and disruption of established biofilms (FIG. 12 and FIG. 13). Peptoids exert direct bactericidal/fungicidal activity against the cell membrane, making retention of potency possible even when microbes are metabolically inactive, often associated with pathogen biofilms.

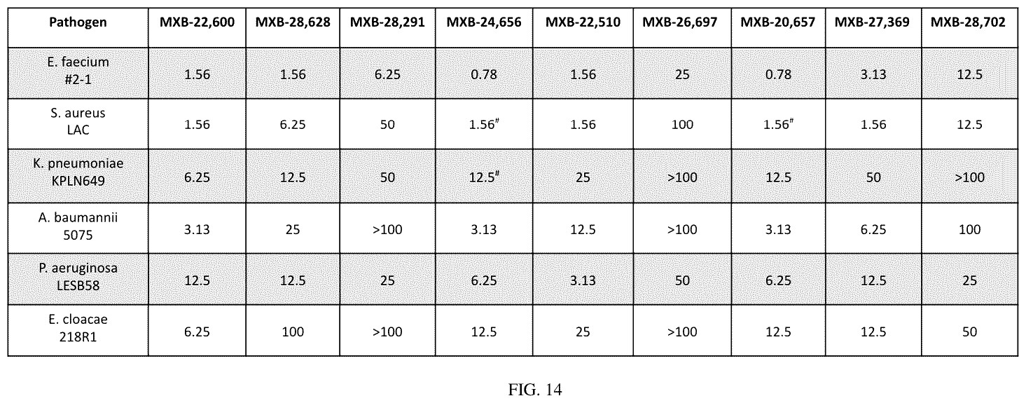

This also includes ESKAPE pathogens, (FIG. 14). The peptoids exert direct bactericidal/fungicidal activity against the cell membrane, making retention of potency possible even when microbes are metabolically inactive, often associated with pathogen biofilms.

### Example 5. Example Formulations of Peptoid Compounds

The peptoids described herein may be formulated into a wide variety of dosage forms for topical, targeted local delivery, or systemic delivery as may be required for oral hygiene administration.

### Topical Formulation Approaches

Topical formulation approaches include powder, solution, suspension, semisolids, or infused into a bandage or other dressing material. Powder formulations include, but are not limited to, powder, granulation, pellets, or mini tablets. These powder dosage forms may be packaged or contained in a simple stick pack, sachet, vial, spray, shaker bottle, or multi-use bottle. The solution formulations may be provided as a solution, granules or powder for reconstitution, disintegrating tablet for dissolution and reconstitution, or incorporated into a spray bottle, with or without materials to provide a scaffold or topical bandage. The suspension formulations include, but are not limited to, aqueous suspension, suspension in another solvent, granules or powder for suspension, or disintegrating tablet for resuspension. These suspension dosage forms may be packaged or contained in a simple stick pack, sachet, vial, shaker bottle, or multi-use bottle. Semisolid formulation approaches include, but are not limited to, creme, gel, ointment, lotion, paste, balm, salve, emulsion, suppository (e.g. embedded in wax or polymer that liquifies at body temperature), spray, including spray on bandages, foam, including spray on foams, or film. Peptoids can also be infused into medical dressings including gauze, bandages, among others. Peptoids may be administered using a disintegrating tablet, drug eluting tablet or tablets, drug eluting beads or granules, or implantable, self dissolving sheet, wafer, block or suppository (e.g. embedded in wax or polymer that liquifies at body temperature), or thin wafer inserted for drug elution at the local site.

### Other Delivery Methods

In addition to topical formulation approaches, alternate administration approaches of the peptoids may be employed. These approaches include injection for local delivery, injection for systemic delivery, transdermal patch or other transdermal approach, intravenous, intranasal, intraocular, aural delivery, sublingual, buccal, or oral delivery, including immediate-release dosage forms, as well as modified release. Injection delivery methods may include intraperitoneal, subcutaneous, intramuscular, intrathecal, or intravenous.

### Example Powder Formulation

The peptoids may be administered as a simple powder as a standalone drug or with additional excipients to improve flowability or other processing requirements. This powder may be filled into a hard gelatin capsule and subsequently filled into bottles, or packaged in a sachet, stick pack, vial, or other container to aid in portability and ease of administration. This powder formulation may be applied directly, or dissolved in an aqueous vehicle for topical administration. A powder for topical administration may be prepared using the following formula:

### Formulation 1

Drug Substance—600 g (active ingredient)
Microcrystalline cellulose—100 g (processing aid—flowability)
Lactose—300 g (processing aid—flowability)
Step 1—blend the microcrystalline cellulose and lactose in a suitable blender and blend for 10 minutes
Step 2—add the drug substance and blend for an additional 10 minutes, or until the drug is uniformly distributed throughout the blender.
Step 3—discharge the powder blend from the blender into a suitable bin or container to store until the filling operation.
Step 4—fill the appropriate amount of blend into each package for storage, transfer, and administration.

### Example 2—Granulation

The drug substance powder can also be incorporated into a granulation that can produce a particle with improved flowability and density relative to the powder in Example 1. This granulation can be prepared either dry, or in the presence of water or other solvent. The binder may be added either wet (in the granulation solution) or dry with the rest of the materials. If water or other solvent is used, the blend is dried in a suitable pharmaceutical drier, such as a vacuum oven, forced air oven, or fluid bed drier. This granulation may be filled into a hard gelatin capsule and subsequently filled into bottles, or packaged in a sachet, stick pack, vial, or other container to aid in portability and ease of administration. This granulation formulation may be applied directly, or dissolved in an aqueous vehicle for topical administration. A granule formulation that may be used for topical application or dissolution into a topical solution may be prepared using the following formula:

### Formulation 2

Drug substance—700 g (active ingredient)
Microcrystalline cellulose—200 g (diluent and processing aid)
Povidone—100 g (binder)
Water (processing aid; removed during processing)
Step 1—add the active and excipients to a suitable pharmaceutical mixer or granulator such as a planetary mixer, high-shear granulator, fluid bed granulator, or extruder.
Step 2—slowly add the water while the mixer is operating until all the water has been added.
Step 3—continue the granulation step until the granulation endpoint is achieved.
Step 4—discharge the wet mass into a container suitable to hold the material until drying.
Step 5—charge the wet mass into a suitable drier and dry until the endpoint of less than 2% water is reached.
Step 6—discharge the dried granulation into a suitable bin or container to store until the filling operation.
Step 7—fill the appropriate amount of granulation into each package for storage, transfer, and administration.

Additional formulation examples are shown below, and follow a similar procedure for preparation.

### Formulation 3

Drug substance—700 g (active ingredient)
Microcrystalline cellulose—275 g (diluent and processing aid)
Povidone—75 g (binder)
Croscarmellose sodium—50 g (disintegrant)
Water (processing aid; removed during processing)

### Formulation 4

Drug substance—600 g (active ingredient)
Microcrystalline cellulose—300 g (diluent and processing aid)
Hydroxypropyl cellulose—100 g (binder)
Water (processing aid; removed during processing)

### Formulation 5

Drug substance—800 g (active ingredient)
Microcrystalline cellulose—125 g (diluent and processing aid)
Hydroxypropyl cellulose—50 g (binder)
Croscarmellose sodium—25 g (disintegrant)
Water (processing aid; removed during processing)

### Example 3—Pellets

The drug substance powder can also be incorporated into a pellet that can produce a particle with improved flowability and density relative to the powder in Example 1, and better flowability than the granulations in Example 2. These pellets may be filled into a hard gelatin capsule and subsequently filled into bottles, or packaged in a sachet, stick pack, vial, or other container to aid in portability and ease of administration. Pellet formulations that may be used for topical application or dissolution into a topical solution may be prepared using similar formulations to those shown in Example 2, with the addition of 2 processing steps. This pellet formulation may be applied directly, or dissolved in an aqueous vehicle for topical administration. Pellet formulation examples are shown here:

### Formulation 6

Drug substance—700 g (active ingredient)
Microcrystalline cellulose—200 g (diluent and processing aid)
Povidone—100 g (binder)
Water (processing aid; removed during processing)

### Formulation 7

Drug substance—700 g (active ingredient)
Microcrystalline cellulose—275 g (diluent and processing aid)
Povidone—75 g (binder)
Croscarmellose sodium—50 g (disintegrant)
Water (processing aid; removed during processing)

### Formulation 8

Drug substance—600 g (active ingredient)
Microcrystalline cellulose—300 g (diluent and processing aid)
Hydroxypropyl cellulose—100 g (binder)
Water (processing aid; removed during processing)

### Formulation 9

Drug substance—800 g (active ingredient)
Microcrystalline cellulose—125 g (diluent and processing aid)
Hydroxypropyl cellulose—50 g (binder)
Croscarmellose sodium—25 g (disintegrant)
Water (processing aid; removed during processing)
Step 1—add the active and excipients to a suitable pharmaceutical mixer or granulator such as a planetary mixer, high-shear granulator, fluid bed granulator, or extruder.
Step 2—slowly add the water while the mixer is operating until all the water has been added.
Step 3—continue the granulation step until the granulation endpoint is achieved.
Step 4—Load the wet mass into a suitable extruder and extrude using a screen with apertures between 300 μm and 800 μm.
Step 5—extrude the wet mass and introduce the extrudate into the marumerizer for pellet formation and spheronization.
Step 6—discharge the wet mass of pellets into a container suitable to hold the material until drying.
Step 7—charge the wet mass into a suitable drier and dry until the endpoint of less than 2% water is reached.
Step 8—discharge the dried pellets into a suitable bin or container to store until the filling operation.
Step 9—fill the appropriate amount of pellets into each package for storage, transfer, and administration.

### Example 4—Mini Tablets

The drug substance powder can also be incorporated into minitablets that can produce a particle with similar performance characteristics as a pellet. Minitablets offer another dry formulation approach, where a solvent may not be required. These minitablets typically have a diameter on the order of 500-2000 μm may be filled into a hard gelatin capsule and subsequently filled into bottles, or packaged in a sachet, stick pack, vial, or other container to aid in portability and ease of administration. This minitablet formulations may be applied directly, or dissolved in an aqueous vehicle for topical administration. Minitablet formulation examples are shown here:

### Formulation 10

Drug substance—600 g (active ingredient)
Microcrystalline cellulose—300 g (diluent)
Lactose—100 g (diluent)
Hypromellose (HPMC)—50 g (binder)
Colloidal silicon dioxide—50 g (glidant)
Magnesium stearate—5 g (lubricant)

### Formulation 11

Drug substance—600 g (active ingredient)
Silicified microcrystalline cellulose—400 g (diluent)
Hypromellose (HPMC)—50 g (binder)
Colloidal silicon dioxide—50 g (glidant)
Magnesium stearate—5 g (lubricant)

### Formulation 12

Drug substance—600 g (active ingredient)
Silicified microcrystalline cellulose—400 g (diluent)
Polyvinylpyrrolidone (PVP)—50 g (binder)
Colloidal silicon dioxide—50 g (glidant)
Magnesium stearate—5 g (lubricant)

### Formulation 13

Drug substance—700 g (active ingredient)
Silicified microcrystalline cellulose—250 g (diluent)
Polyethylene glycol (PEG)—50 g (binder)
Colloidal silicon dioxide—50 g (glidant)
Magnesium stearate—5 g (lubricant)

### Example 7—Cream

A topical cream formulation may be prepared using the following formula for the preparation of a batch of approximately 1 Kg:

### Formulation 14

Methyl paraben—0.25 g (preservative)
Propyl paraben—0.15 g (preservative)
Polysorbate 60—10 g (emulsifier)
Propylene glycol—120 g (viscosity modifier)
Stearyl alcohol—200 g (oleaginous phase)
White petrolatum—200 g (oleaginous phase)
Purified water—470 g (aqueous base)
Step 1—mix the stearyl alcohol and white petrolatum and heat to approximately 75° C. to melt the base.
Step 2—Dissolve the remaining excipients in the purified water by stirring until a solution is obtained.
Step 3—Add approximately 5 g of drug substance to the purified water solution and mix for 5 additional minutes to dissolve the drug to manufacture a 5% ointment.
Step 4—Slowly incorporate the aqueous solution to the oleaginous base and mix until it is well mixed.
Step 5—Fill the cream into a suitable package such as a tube or pump bottle.

### Example 8—Gel

Topical gels can be used for sustained-release of actives, provide lubrication, and a carrier of pharmaceutical agents. Hydrogels are water-based and are less oily than creams or ointments, as well as exhibit excellent spreading properties, and may exhibit a higher retention time on the skin. Hydrogels can be simple formulations and may provide for a higher drug capacity than oil based formulations due to the high aqueous solubility of the drug substance. Gel formulation examples are shown here:

### Formulation 15

Drug Substance—50 g (active)
Carbopol—300 g (polymer/viscosity)
Purified Water—650 g (solvent)

### Formulation 16

Drug Substance—50 g (active)
Sodium carboxymethylcellulose—400 g (polymer/viscosity)
Purified Water—550 g (solvent)

### Formulation 17

Drug Substance—50 g (active)
Hypromellose—200 g (polymer/viscosity)
Purified Water—750 g (solvent)
Step 1—Slowly add the polymer to the purified water while stirring slowly using a suitable mixer such as a Silverson mixer. Continue to mix until the polymer exhibits a lump-free dispersion.
Step 2—Slowly add the drug substance to the polymer dispersion and mix until dissolved.
Step 3—Fill the gel into a suitable package such as a tube or pump bottle.

### Example 9—Ointment

Hydrophilic ointment may be prepared using the following formula for the preparation of about 1 Kg of base:

### Formulation 18

Methyl paraben—0.25 g (preservative)
Propyl paraben—0.15 g (preservative)
Sodium lauryl sulfate—10 g (emulsifier)
Propylene glycol—120 g (viscosity modifier)
Stearyl alcohol—250 g (oleaginous phase)
White petrolatum—250 g (oleaginous phase)
Purified water—370 g (aqueous base)
Step 1—mix the stearyl alcohol and white petrolatum and heat to approximately 75° C. to melt the base.
Step 2—Dissolve the remaining excipients in the purified water by stirring until a solution is obtained.
Step 3—Add approximately 1 g of drug substance to the purified water solution and mix for 5 additional minutes to dissolve the drug to manufacture a 1% ointment.
Step 4—Slowly incorporate the aqueous solution to the oleaginous base and mix until it congeals.
Step 5—Fill the ointment into a suitable package.

### Example 10—Sterile Solution for Subcutaneous or Intramuscular Administration

The drug may be incorporated into a solution for delivery via intramuscular (IM), subcutaneous (SC), or intravenous (IV) administration. Formulations designed to deliver active drug substances via the IM or SC route will generally have similar concentrations and volumes of administration. Formulations intended to provide 1 L of drug formulation for SC or IM administration are shown here:

Formulation 19—10% (100 mg/mL)
Drug Substance—100 g (active)
Phosphate Buffer solution—1 L (solvent)
pH adjustment—0.1 N NaOH or 0.1 N HCl (pH adjustment)
   Formulation 20—5% (50 mg/mL)
Drug Substance—50 g (active)
Phosphate Buffer solution—1 L (solvent)
pH adjustment—0.1 N NaOH or 0.1 N HCl (pH adjustment)
   Formulation 21—1% (10 mg/mL)
Drug Substance—10 g (active)
Phosphate Buffer solution—1 L (solvent)
pH adjustment—0.1 N NaOH or 0.1 N HCl (pH adjustment)
   Formulation 22—0.1% (1 mg/mL)
Drug Substance—1 g (active)
Phosphate Buffer solution—1 L (solvent)
pH adjustment—0.1 N NaOH or 0.1 N HCl (pH adjustment)
   Formulation 23—0.05% (500 μg/mL)
Drug Substance—0.5 g (active)
Phosphate Buffer solution—1 L (solvent)
pH adjustment—0.1 N NaOH or 0.1 N HCl (pH adjustment)
Step 1—Slowly add the drug substance to the buffer solution while stirring.
Step 2—Continue stirring until a clear solution is obtained.
Step 3—Measure the pH of the solution, and adjust to a pH of 6.5-7.5 using the dilute HCl or NaOH solution.

Step 4—Sterile filtration using a 0.22 μm filter, and fill into a sterile syringe for a pre-filled syringe drug-device combination.

The above disclosure contains various examples of peptoid compound compositions and methods of use thereof. Aspects of these various examples may all be combined with one another, even if not expressly combined in the present disclosure, unless they are clearly mutually exclusive.

In addition, various example materials are discussed herein and are identified as examples, as suitable materials, and as materials included within a more generally described type of material, for example by use of the term “including” or “such-as.” All such terms are used without limitation, such that other materials falling within the same general type exemplified but not expressly identified may be used in the present disclosure as well.

Furthermore, unless it is otherwise clear that a single entity is intended, terms such as “a,” “an,” and “the” are not intended to refer to only a singular entity and include the general class of which a specific example is described for illustration. In addition, unless it is clear that a precise value is intended, numbers recited herein should be interpreted to include variations above and below that number that may achieve substantially the same results as that number, or variations that are “about” the same number. Finally, a derivative as disclosed herein may include a chemically modified molecule that has an addition, removal, or substitution of a chemical moiety of the parent molecule.

It is understood the use of the alternative (e.g., “or”) herein is taken to mean either one or both or any combination thereof of the alternatives. The term “and/or” used herein is to be taken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

Various compositions may be identified by trade name in this application. All such trade names refer to the relevant composition or instrument as it existed as of the earliest filing date of this application, or the last date a product was sold commercially under such trade name, whichever is later. One of ordinary skill in the art will appreciate that variant compositions and instruments sold under the trade name at different times will typically also be suitable for the same uses.

The above disclosed subject matter is to be considered illustrative, and not restrictive, and the appended claims are intended to cover all such modifications, enhancements, and other implementations which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A composition for use in preventing or treating an oral infection in a subject, the composition comprising one or more peptoid compounds in an amount effective to prevent or treat the oral infection.

2. The composition of claim 1, wherein the one or more peptoids are selected from:
a peptoid compound H-Ntridec-NLys-Nspe-Nspe-NLys-NH₂;
a peptoid compound H-NLys-NSpe(p-Br)-NSpe(p-Br)-NLys-NSpe(p-Br)5-NSpe(p-Br)-NH₂; and
a combination thereof;
wherein each peptoid is in an amount of effective to prevent or treat the oral infection.

3. The composition of claim 1, wherein the oral infection is an infection of the oral cavity, gums, lips, teeth, or any combination thereof.

4. The composition of claim 1, wherein the infection is an infection of an unidentified pathogen.

5. The composition of claim 1, wherein the infection is a bacterial infection, a fungal infection, a viral infection, or any combinations thereof.

6. The composition of claim 1, wherein the infection is an infection of a pathogen selected from Herpes Simplex Virus-1 (HSV-1), Streptococcus mutans, Candida albicans, Porphyromonas gingivalis, and any combinations thereof.

7. The composition of claim 1, wherein the composition is effective to prevent, decrease, or inhibit decrease a biofilm associated with the oral infection.

8. The composition of claim 1, formulated for topical administration, transdermal administration, transmucosal administration, subcutaneous administration, intramuscular administration, or intravenous administration to the subject.

9. The composition of claim 1, formulated as a sublingual tablet, a gum, a lozenge, a thin film, a spray, a rinse, a toothpaste, an ointment, a creme, a balm, or any combinations thereof.

10. The composition of claim 1, wherein the effective amount is from 1-1000 mg/day, 25-750 mg/day, 50-500 mg/day, or 100-400 mg/day.

11. The composition of claim 1, formulated for administration one, two, three, or four times per day, once per week, once every two weeks, or once per month.

12. A method of preventing or treating an oral infection in a subject, the method comprising:
administering the composition of claim 1, in an amount effective to prevent or treat the oral infection.
